(19) United States
(12) Patent Application Publication
(10) Pub. No.: US 2026/0204352 A1
(43) Pub. Date: Jul. 16, 2026

(54) METHODS FOR DISTINGUISHING ANEUPLODIES IN NON-INVASIVE PRENATAL TESTING

(71) Applicant: THE JOHNS HOPKINS UNIVERSITY, Baltimore, MD (US)

(72) Inventors: Daniel ARIAD, Baltimore, MD (US); Manuel VIOTTI, Foster City, CA (US); Rajiv MCCOY, Baltimore, MD (US)

(21) Appl. No.: 19/135,464

(22) PCT Filed: Nov. 28, 2023
(86) PCT No.: PCT/US2023/081262

§ 371 Date: Jun. 4, 2025

Related U.S. Application Data
(60) Provisional application No. 63/387,770, filed on Dec. 16, 2022.

(51) Int. Cl. G16B 30/10; G16B 20/20

(52) U.S. Cl. CPC G16B 30/10; G16B 20/20

(57) ABSTRACT

Provided herein are methods of non-invasive prenatal testing to distinguish between meiotic- and mitotic-origin aneuploidies in certain embodiments. Related systems and computer program products are also provided. In some embodiments, it includes methods for distinguishing between meiotic- and mitotic-origin aneuploidies of a subject, such as an in utero fetus, using non-invasive prenatal genetic testing. In some embodiments, it includes a computational statistical method of distinguishing between meiotic- and mitotic-origin aneuploidies of a subject.

### CROSS-REFERENCE TO RELATED APPLICATIONS

This application is the national stage entry of International Patent Application No. PCT/US2023/081262, filed on Nov. 28, 2023, and published as WO 2024/129354 A1 on Jun. 20, 2024, which claims the benefit of U.S. Provisional Application No. 63/387,770, filed on 16 Dec. 2022, which are hereby incorporated by reference herein in their entireties.

### STATEMENT OF GOVERNMENT INTEREST

This invention was made with government support under contract GM133747 awarded by the National Institutes of Health. The government has certain rights in the invention.

### BACKGROUND

Whole-chromosome gains and losses (aneuploidies) are extremely common in human embryos and are the leading cause of pregnancy loss and congenital disorders, both in the context of in vitro fertilization (IVF) and natural conception. Aneuploidy may occur due to chromosome segregation errors in meiosis (egg or sperm formation) or mitosis (post-fertilization cell division). Aneuploidy frequently arises during maternal meiosis due to mechanisms such as classical non-disjunction, premature separation of sister chromatids (PSSC), and reverse segregation (RS). Such meiotic aneuploidies are strongly associated with maternal age, with risk of aneuploid conception increasing exponentially starting around age 35.

Though less well understood, recent work has also demonstrated that aneuploidy of mitotic origin is particularly prevalent during the initial post-zygotic cell divisions, potentially permitted by relaxation of cell cycle checkpoints prior to embryonic genome activation. Such mitotic errors, which appear to be independent of maternal or paternal age, can generate mosaic embryos possessing both normal and aneuploid cells. Mechanisms of mitotic aneuploidy include anaphase lag and mitotic non-disjunction, but also novel phenomena such as multipolar mitotic division, whereby the diploid genome is partitioned among three or more daughter cells, resulting in massive chromosome loss. Such abnormal mitotic divisions are surprisingly common in cleavage-stage embryos and may largely explain the high observed rates of embryonic mortality (~50%) during preimplantation human development.

While uniform aneuploidies arising from meiotic errors are unambiguously harmful, mosaic aneuploidies of mitotic origin are potentially compatible with healthy live birth. Moreover, low-level mosaic aneuploidy may be more prevalent than previously appreciated, systematically underestimated due to the reliance on biopsies of one or few cells that are used for pre-implantation genetic testing for aneuploidy (PGT-A).

PGT-A seeks to rank in vitro fertilized embryos for transfer based on their inferred ploidy statuses. LD-PGTA, a method disclosed for example in PCT Publication No. WO 2022/098980, entitled Methods and Related Aspects for Analyzing Chromosome Number Status, the content of which is incorporated by reference in its entirety herein, attempts to distinguish these error mechanisms using low-coverage sequencing data from PGT-A.

In addition to or in the alternative to PGT-A testing, post-implantation genetic testing, i.e., after either IVF or natural conception, is also available to pregnant women to assist in the detection of fetal aneuploidy. One such test is non-invasive prenatal genetic testing (NIPT), which relies on the use of a maternal blood sample. NIPT is based on the observation that cell-free fetal DNA (cffDNA) that originates from placental trophoblast cells circulates freely in maternal blood (Lo, Y. D. et al., Presence of fetal DNA in maternal plasma and serum, The Lancet 1997, 350(9076):485-87). By extracting and assaying the chromosomal constitution of this cffDNA, NIPT may be capable of detecting common chromosome abnormalities such as Trisomy 21 (the genetic cause of Down Syndrome), Trisomy 18 (the genetic cause of Edwards' Syndrome), and Trisomy 13 (the genetic cause of Patau Syndrome), as well as fetal sex and various sex chromosome aneuploidies. NIPT can be applied starting at week 10 of pregnancy, with little risk to the mother or fetus. Since its introduction more than a decade ago, the accuracy of NIPT has steadily increased, and the test has been deployed across a large and growing patient population.

Nevertheless, NIPT still carries a risk of false positive diagnoses. When NIPT results suggest the potential existence of a chromosome abnormality, more invasive diagnostic tests such as amniocentesis, chorionic villus sampling (CVS), and cordocentesis may then be recommended to confirm the result. Unfortunately, amniocentesis can only be performed after 15 weeks of gestation and carries a risk of 0.6-0.86% for miscarriage (Eddleman, K. A. et al., Pregnancy loss rates after midtrimester amniocentesis, Obstetrics and Gynecology 2006, 108(5):1067-72; Wilson, R. D. et al., Mid-trimester amniocentesis fetal loss rate, J. Obstetrics Gynaecology Canada 2007, 29(7):586-90). Similarly, cordocentesis is recommended only after 10 weeks of gestation and carries a fetal loss rate of 1.4-1.9% (Ghidini, A. et al., Complications of fetal blood sampling, Am. J. Obstetrics Gynecology 1993, 168(5):1339-44; Tongsong, T. et al., Second-trimester cordocentesis and the risk of small for gestational age and preterm birth, Obstetrics and Gynecology 2014, 124(5):919-25).

By considering amniocentesis results as ground truth, meta-analyses have demonstrated that NIPT applied to trisomies of chromosomes 13, 18 and 21 achieve detection rates above 95% with false positive rates (FPR) below 0.1%. See Taylor-Phillips, S. et al., Accuracy of non-invasive prenatal testing using cell-free DNA for detection of Down, Edwards and Patau syndromes: A systematic review and meta-analysis, BMJ 2016, 6(1):e010002; Iwarsson, E. et al., Analysis of cell-free fetal DNA in maternal blood for detection of trisomy 21, 18 and 13 in a general pregnant population and in a high risk population—a systematic review and meta-analysis, Acta Obstetricia et Gynecologica Scandinavica 2017, 96(1):7-18; Mackie, F. et al., The accuracy of cell-free fetal DNA-based non-invasive prenatal testing in singleton pregnancies: A systematic review and bivariate meta-analysis, Int'l J. Obstetrics & Gynaecology 2017, 124(1):32-46; Gil, M et al., Analysis of cell-free DNA in maternal blood in screening for aneuploidies: Updated meta-analysis, Ultrasound in Obstetrics & Gynecology 2017, 50(3):302-314. NIPT applied to sex chromosome aneuploidies achieves detection rates above 90% with FPRs below 0.3%. See Mazloom, A. R. et al., Noninvasive prenatal detection of sex chromosomal aneuploidies by sequencing circulating cell-free DNA from maternal plasma, Prenatal diagnosis 2013, 33(6):591-97; Samango-Sprouse, C. et al., SNP-based non-invasive prenatal testing detects sex chromosome aneuploidies with high accuracy, Prenatal Diagnosis 2013, 33(7):643-49; Hooks, J. et al., Non-invasive risk assessment of fetal sex chromosome aneuploidy through directed analysis and incorporation of fetal fraction, Prenatal diagnosis 2014, 34(5):496-99.

In addition to technical artifacts, one of the main causes of discordance between NIPT and amniocentesis results is a phenomenon called confined placental mosaicism (CPM), whereby cells of the placenta possess different chromosomal content than cells of the fetus, confounding interpretation. Other biological phenomena such as vanishing twin syndrome, maternal cancers, or maternal chromosome abnormalities can similarly confound NIPT results, as the observed chromosome abnormalities may be incorrectly attributed to the fetus (Curnow, K. J. et al., Detection of triploid, molar, and vanishing twin pregnancies by a single-nucleotide polymorphism-based noninvasive prenatal test, Am. J. Obstetrics Gynecology 2015, 212(1):79-e1; Amant, F. et al., Presymptomatic identification of cancers in pregnant women during noninvasive prenatal testing, JAMA Oncology 2015, 1(6):814-19; Wang, Y. et al., Maternal mosaicism is a significant contributor to discordant sex chromosomal aneuploidies associated with noninvasive prenatal testing, Clinical Chem 2014, 60(1):251-259).

Accordingly, there is a desire for more accurate methods of conducting NIPT to further reduce false positive results of aneuploidy. The ability to distinguish meiotic- and mitotic-origin aneuploidies after conception may thus prove valuable for increasing the accuracy of NIPT.

### SUMMARY

The present disclosure relates, in certain aspects, to methods for distinguishing between meiotic- and mitotic-origin aneuploidies of a subject, such as an in utero fetus, using non-invasive prenatal genetic testing.

In one aspect, the present disclosure provides a computational statistical method of distinguishing between meiotic- and mitotic-origin aneuploidies of a subject. In certain embodiments, disclosed herein is a method of distinguishing between meiotic- and mitotic-origin aneuploidies of a subject at least partially using a computer, the method comprising: (1) obtaining NIPT sequencing data from a sample from the subject's mother, the sample comprising cell-free fetal DNA (cffDNA) nucleotide fragments originating from the subject and cell-free maternal DNA (cfmDNA) nucleotide fragments originating from the mother; (2) probabilistic separation, by the computer, of cffDNA nucleotide fragments in the sample from the cfmDNA nucleotide fragments based on at least one distinguishing characteristic (e.g., aligned coordinates of reads relative to the nearest nucleosome, inferred fragments lengths, methylation patterns, and/or patterns at fragment edges (blunt vs. jagged ends)); (3) identifying sequencing reads from the cffDNA nucleotide fragments that comprise sequence information from an aneuploid chromosome; (4) dividing the sequencing reads from the aneuploid chromosome into a plurality of linkage disequilibrium (LD) blocks or genomic windows (GWs); (5) selecting one or more of the sequencing reads corresponding to one or more of the plurality of LD blocks or GWs to produce a set of selected sequencing reads; (6) determining probabilities of observing the selected set of sequencing reads under a meiotic-origin model and under a mitotic-origin model, for example given knowledge of haplotype structure from an external reference panel of phased genome sequences, to produce a set of probability data; and (7) aggregating log-likelihood ratios across the plurality of LD blocks or GWs to produce an aggregated log-likelihood ratio using the set of probability data, wherein an aggregated log-likelihood ratio significantly greater than zero indicates that the aneuploid chromosome is a meiotic-origin aneuploidy, and wherein an aggregated log-likelihood ratio significantly less than zero indicates that the aneuploid chromosome is a mitotic-origin aneuploidy, thereby distinguishing between the meiotic- and the mitotic-origin aneuploidies.

In certain embodiments, the method disclosed herein further comprises sequencing the cfmDNA nucleotide fragments or obtaining sequenced cfmDNA nucleotide fragments, and in certain embodiments, the method disclosed herein further comprises obtaining a phased reference haplotype panel, such as obtaining joint frequencies of corresponding single nucleotide polymorphs (SNPs) from a phased reference haplotype panel, such as a phased reference haplotype panel obtained from sequenced cfmDNA nucleotide fragments. In certain embodiments disclosed herein, the set of selected sequencing reads comprises overlapping informative SNPs that tag common haplotype variation. In certain embodiments disclosed herein, the phased reference haplotype panel is obtained from a population reference panel.

In various embodiments, the methods disclosed herein comprise at least partially using a computer. In certain embodiments, the method comprises obtaining, by the computer, non-invasive prenatal testing (NIPT) sequencing data from a sample from the subject's mother, the sample comprising cell-free fetal DNA (cffDNA) nucleotide fragments originating from the subject and cell-free maternal DNA (cfmDNA) nucleotide fragments originating from the mother and identifying, via probabilistic separation using the computer, the cffDNA nucleotide fragments in the sample from the cfmDNA nucleotide fragments based on at least one distinguishing characteristic. In certain embodiments, the sequencing reads from the cffDNA nucleotide fragments that comprise sequence information from an aneuploid chromosome are received by a computer, and in certain embodiments, the sequencing reads from the aneuploid chromosome are divided by the computer into the plurality of LD blocks or GWs.

In certain embodiments of the methods disclosed herein, the sample from the subject's mother is a blood serum sample, and in certain embodiments, the in utero fetus has a gestational age ranging from about 6 weeks to about 40 weeks, such as, for example, greater than about or about 6 weeks, greater than about or about 7 weeks, greater than about or about 8 weeks, greater than about or about 9 weeks, greater than about or about 10 weeks, greater than about or about 11 weeks, greater than about or about 12 weeks, greater than about or about 13 weeks, or greater than about or about 14 weeks.

In certain embodiments of the methods disclosed herein, the aneuploid chromosome comprises a trisomy. In certain embodiments, the methods disclosed herein further comprise determining whether an aneuploidy is due to a meiosis I error or a meiosis II error when the aneuploid chromosome is a meiotic-origin aneuploidy. In certain embodiments, the methods disclosed herein further comprise determining a significance of the aggregated log-likelihood ratio using at least one statistical procedure, such as a bootstrap or weighted jackknife procedure.

In certain embodiments of the methods disclosed herein, the sequencing reads from the cffDNA nucleotide fragments comprise a coverage of less than about 2×, less than about 1×, less than about 0.50×, less than about 0.25×, less than about 0.15×, less than about 0.10×, or less than about 0.05× of a genome of a fetus. In certain embodiments, a probability that two sequencing reads are obtained from an identical haplotype under the meiotic-origin model is about ⅓, and in certain embodiments, a probability that two sequencing reads are obtained from an identical haplotype under the mitotic-origin model is about 5/9.

In another aspect, disclosed herein is a system comprising at least one controller that comprises, or is capable of accessing, computer readable media comprising non-transitory computer executable instructions which, when executed by at least one electronic processor, perform at least: (1) receiving NIPT sequencing data from a sample from the subject's mother, the sample comprising cffDNA nucleotide fragments originating from the subject and cfmDNA nucleotide fragments originating from the mother; (2) identifying, via probabilistic separation, cffDNA nucleotide fragments in the sample from the cfmDNA nucleotide fragments based on at least one distinguishing characteristic; (3) receiving sequencing reads obtained cell-free fetal DNA (cffDNA) nucleotide fragments comprising sequence information from an aneuploid chromosome; (4) dividing the sequence information from the aneuploid chromosome into a plurality of linkage disequilibrium (LD) blocks or genomic windows (GWs); (5) selecting one or more of the sequencing reads corresponding to one or more of the plurality of LD blocks or GWs to produce a set of selected sequencing reads; (6) determining probabilities of observing the selected set of sequencing reads under a meiotic-origin model and under a mitotic-origin model, for example given knowledge of haplotype structure from an external reference panel of phased genome sequences, to produce a set of probability data; and (7) aggregating log-likelihood ratios across the plurality of LD blocks or GWs to produce aggregated log-likelihood ratio using the set of probability data, wherein an aggregated log-likelihood ratio significantly greater than zero indicates that the aneuploid chromosome is a meiotic-origin aneuploidy, and wherein an aggregated log-likelihood ratio significantly less than zero indicates that the aneuploid chromosome is a mitotic-origin aneuploidy.

In another aspect, disclosed herein is computer readable media comprising non-transitory computer executable instructions which, when executed by at least electronic processor, perform at least: (1) receiving NIPT sequencing data from a sample from the subject's mother, the sample comprising cffDNA nucleotide fragments originating from the subject and cfmDNA nucleotide fragments originating from the mother; (2) identifying, via probabilistic separation, cffDNA nucleotide fragments in the sample from the cfmDNA nucleotide fragments based on at least one distinguishing characteristic; (3) receiving sequencing reads obtained cffDNA nucleotide fragments comprising sequence information from an aneuploid chromosome; (4) dividing the sequence information from the aneuploid chromosome into a plurality of linkage disequilibrium (LD) blocks or genomic windows (GWs); (5) selecting one or more of the sequencing reads corresponding to one or more of the plurality of LD blocks or GWs to produce a set of selected sequencing reads; (6) determining probabilities of observing the selected set of sequencing reads under a meiotic-origin model and under a mitotic-origin model, for example given knowledge of haplotype structure from an external reference panel of phased genome sequences, to produce a set of probability data; and (7) aggregating log-likelihood ratios across the plurality of LD blocks or GWs to produce aggregated log-likelihood ratio using the set of probability data, wherein an aggregated log-likelihood ratio significantly greater than zero indicates that the aneuploid chromosome is a meiotic-origin aneuploidy, and wherein an aggregated log-likelihood ratio significantly less than zero indicates that the aneuploid chromosome is a mitotic-origin aneuploidy.

In certain embodiments of the system or computer readable media disclosed herein, the cffDNA originated from a sample, such as a maternal blood sample, comprising the cffDNA and cfmDNA. In certain embodiments of the system or computer readable media disclosed herein, instructions further perform obtaining joint frequencies of corresponding SNPs from a phased reference haplotype panel, and in certain embodiments, the phased reference haplotype panel is obtained from cfmDNA, wherein the cfmDNA originated from a sample comprising both the cffDNA and cfmDNA, such as a maternal blood sample.

### BRIEF DESCRIPTION OF THE DRAWINGS

The accompanying drawings, which are incorporated in and constitute a part of this specification, illustrate certain embodiments, and together with the written description, serve to explain certain principles of the methods, systems, and related computer readable media disclosed herein. The description provided herein is better understood when read in conjunction with the accompanying drawings which are included by way of example and not by way of limitation. It will be understood that like reference numerals identify like components throughout the drawings, unless the context indicates otherwise. It will also be understood that some or all of the figures may be schematic representations for purposes of illustration and do not necessarily depict the actual relative sizes or locations of the elements shown.

FIG. 1 is a schematic depicting exemplary types of meiotic and mitotic errors and various forms of chromosomal abnormalities with respect to their composition of identical and distinct parental homologs.

FIG. 2 is a flow chart that schematically depicts exemplary method steps according to some aspects disclosed herein.

FIGS. 3A-3D schematically depict exemplary method steps according to some aspects disclosed herein. FIG. 3A illustrates a method step comprising selecting sequence reads, within defined genomic windows, that overlap informative SNPs that tag common haplotype variation, as described herein. FIG. 3B illustrates a method step comprising obtaining joint frequencies of corresponding SNPs from a phased panel of reference haplotypes, such as a phased panel obtained from cfmDNA, as described herein. FIG. 3C illustrates a method step comprising selecting multiple (e.g., 2-16) sequencing reads and computing probabilities of observed alleles, as described herein. FIG. 3D illustrates a method step comprising computing a likelihood ratio and estimating the mean and variance by sub-sampling random sets of reads using a bootstrapping approach, as described herein.

FIG. 4 is a schematic diagram of an exemplary system suitable for use with certain aspects disclosed herein.

FIG. 5A is a graph showing a statistical probability of homolog reads from cffDNA from a meiotic-origin trisomy based on a level of contamination with cfmDNA.

FIG. 5B is a graph showing a statistical probability of homolog reads from cffDNA from a mitotic-origin trisomy based on a level of contamination with cfmDNA.

### DEFINITIONS

In order for the present disclosure to be more readily understood, certain terms are first defined below. Additional definitions for the following terms and other terms may be set forth throughout the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

It is also to be understood that the terminology used herein is for the purpose of describing particular embodiments only and is not intended to be limiting. Further, unless defined otherwise, all technical and scientific terms used herein have the same meaning as commonly understood by one of ordinary skill in the art to which this disclosure pertains. In describing and claiming the methods, computer readable media, systems, and component parts, the following terminology, and grammatical variants thereof, will be used in accordance with the definitions set forth below.

About: As used herein, “about” or “approximately” or “substantially” as applied to one or more values or elements of interest, refers to a value or element that is similar to a stated reference value or element. In certain embodiments, the term “about” or “approximately” or “substantially” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Allele Frequency: As used herein, “allele frequency” refers to the relative frequency of an allele at a particular locus in a population or in a given subject. Allele frequency is typically expressed as a fraction or percentage.

Aneuploidy: As used herein, “aneuploid,” “aneuploidy,” and the like refer to a genetic state wherein the total number of chromosomes in a cell is abnormal for the species. For example, a typical human cell is considered aneuploid if it contains a total of 47 chromosomes (triploid) instead of 46 chromosomes.

Cell-free fetal DNA: As used herein, “cell-free fetal DNA” or “cffDNA” indicates extracellular nucleotide fragments of deoxyribonucleic acid from a fetus that originate from trophoblast cells, which may circulate in maternal blood, and contain genetic information of the fetus.

Cell-free maternal DNA: As used herein, “cell-free maternal DNA” or “cfmDNA” indicates extracellular nucleotide fragments of deoxyribonucleic acid from a pregnant woman and contain genetic information of the woman.

Coverage: As used herein, “coverage” refers to the number of nucleic acid molecules that originate from a particular base position.

Meiotic-origin aneuploidy: As used herein, the term “meiotic-origin aneuploidy” indicates an aneuploidy status of a cell that originates during an error in the meiosis stage of cell division.

Mitotic-origin aneuploidy: As used herein, the term “mitotic-origin aneuploidy” indicates an aneuploidy status of a cell that originates from an error during a mitotic division of the cell cycle.

Next Generation Sequencing: As used herein, “next generation sequencing” or “NGS” refers to sequencing technologies having increased throughput as compared to traditional Sanger- and capillary electrophoresis-based approaches, for example, with the ability to generate hundreds of thousands of relatively small sequence reads at a time. Some examples of next generation sequencing techniques include, but are not limited to, sequencing by synthesis, sequencing by ligation, and sequencing by hybridization.

Probabilistic separation: As used herein, “probabilistic separation” refers to the act of identifying, via the use of probabilities and/or mathematical formulae for example through the use of a computer, at least two distinct entities having at least one distinguishing characteristic. For example, probabilistic separation may in certain embodiments refer to the identification of cffDNA and cfmDNA through the use of computational statistics.

Sequencing: As used herein, “sequencing” refers to any of a number of technologies used to determine the sequence (e.g., the identity and order of monomer units) of a biomolecule, e.g., a nucleic acid such as DNA or RNA. Exemplary sequencing methods include, but are not limited to, targeted sequencing, single molecule real-time sequencing, exon or exome sequencing, intron sequencing, electron microscopy-based sequencing, panel sequencing, transistor-mediated sequencing, direct sequencing, random shotgun sequencing, Sanger dideoxy termination sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, emulsion PCR, co-amplification at lower denaturation temperature-PCR (COLD-PCR), multiplex PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD™ sequencing, MS-PET sequencing, and a combination thereof. In some embodiments, sequencing can be performed by a gene analyzer such as, for example, gene analyzers commercially available from Illumina, Inc., Pacific Biosciences, Inc., or Applied Biosystems/Thermo Fisher Scientific, among many others.

Sequence Information: As used herein, “sequence information” in the context of a nucleic acid polymer means the order and identity of monomer units (e.g., nucleotides, etc.) in that polymer.

Subject: As used herein, “subject” or “test subject” refers to an animal, such as a mammalian species (e.g., human) or avian (e.g., bird) species. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or pathology or a predisposition to the disease or pathology, or an individual that is in need of therapy or suspected of needing therapy. The terms “individual” or “patient” are intended to be interchangeable with “subject.” A “reference subject” refers to a subject known to have or lack specific properties (e.g., known ocular or other pathology and/or the like). In certain instances, a “subject” can include a female, such as a pregnant female, and in certain embodiments, a “subject” can indicate a fetus, such as an in utero fetus.

### DETAILED DESCRIPTION

Extra or missing chromosomes—a phenomenon termed aneuploidy—frequently arises during human meiosis and post-zygotic mitosis and is the leading cause of pregnancy loss, including in the context of both in vitro fertilization (IVF) and natural conception. While meiotic aneuploidies affect all cells and are deleterious, mitotic errors generate mosaicism, which may be compatible with healthy live birth. Distinguishing meiotic and mitotic errors may therefore improve the efficacy of non-invasive prenatal testing (NIPT), which seeks to analyze maternal blood for fetal ploidy statuses and other fetal genetic characteristics.

Disclosed herein is a method of distinguishing between aneuploidies of meiotic origin and aneuploidies of mitotic origin in the fetus of a pregnant woman through the use of non-invasive prenatal testing. The NIPT methods disclosed herein comprise an initial step of identifying and probabilistically separating, through the use of a computer, cell-free fetal DNA (cffDNA) nucleotide sequence fragments from cell-free maternal DNA (cfmDNA) nucleotide sequence fragments; analyzing the cffDNA nucleotide sequence fragments for aneuploidy status, and then distinguishing between aneuploidies of meiotic origin and aneuploidies of mitotic origin.

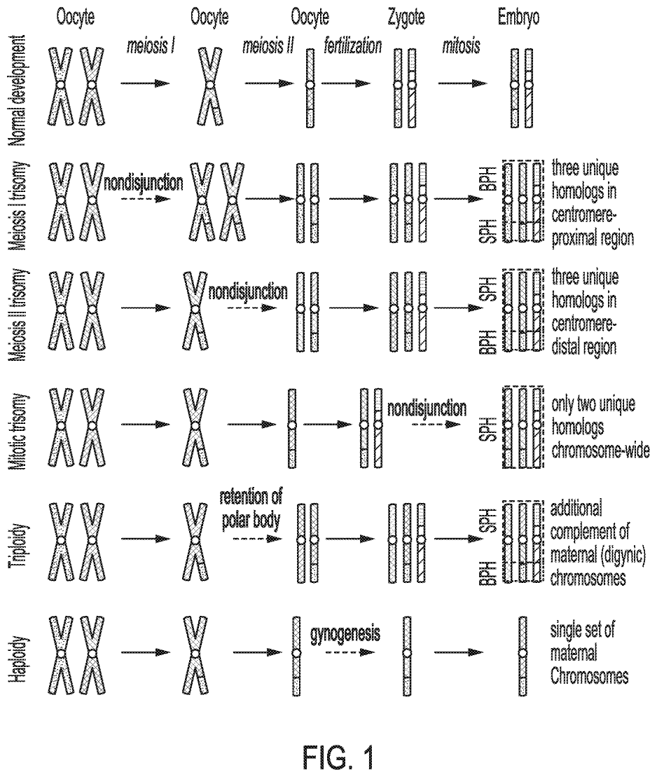

The methods disclosed herein extend the methods of LD-PGTA, as disclosed, for example, in PCT Publication No. WO 2022/098980, to the context of NIPT. By leveraging genotyping signatures from low-coverage sequencing data, LD-PGTA may distinguish harmful trisomies arising via meiosis from those that may have arisen via mitosis. The latter category encompasses cases of confined placental mosaicism (CPM) or maternal cancer, thus mitigating this source of false positives that may result from NIPT. As used herein, low-coverage sequencing PGT-A data indicates that less than about 1/100th of the genome is covered by any sequencing read. In low-coverage sequencing data, aligned reads rarely overlap. Accordingly, through the methods disclosed herein, NIPT may be extended to be used to accurately predict the aneuploid origin of a chromosome. Notably, trisomies of meiotic origin are expected to produce a unique genetic signature characterized by the presence of three unique parental haplotypes (two from a single parent), whereas trisomies of mitotic origin are expected to produce a genetic signature characterized by the presence of only two unique haplotypes chromosome-wide. See, e.g., FIG. 1.

Linkage disequilibrium (LD) is the non-random association of alleles at different loci, such that the frequency of association of the alleles is higher or lower than what would be expected if the loci were independent and associated randomly. One consequence of LD in a population is that observing an allele at one locus in the genome may inform the probability of observing other alleles at loci on the same haplotype. Thus, LD may be used to determine whether two sequencing reads originated from the same or different chromosomes, based on known patterns of linkage between alleles observed on those sequencing reads. It can therefore be determined whether a given sample containing sequencing reads from an aneuploid chromosome contains three unique copies of a chromosome, indicating an aneuploidy of meiotic origin, versus an aneuploid chromosome containing only two unique copies of a chromosome, indicating an aneuploid of mitotic origin.

In certain embodiments, disclosed herein is a computational statistical method of distinguishing between such meiotic- and mitotic-origin aneuploidies of a subject at least partially using a computer, the method comprising (1) obtaining NIPT sequencing data from a sample from the subject's mother that comprises cffDNA nucleotide fragments and cfmDNA nucleotide fragments; (2) identifying, for example probabilistic separation using the computer, the cffDNA nucleotide fragments from the cfmDNA nucleotide fragments based on at least one distinguishing characteristic, as disclosed herein, such as aligned coordinates of reads relative to the nearest nucleosome, inferred fragments lengths, methylation patterns, and/or patterns at fragment edges (blunt vs. jagged ends); (3) identifying sequencing reads from the cffDNA nucleotide fragments that comprise sequence information from an aneuploid chromosome. In certain embodiments, the methods disclosed herein further comprise evaluating whether the aneuploid chromosome is of meiotic- or mitotic-origin by (4) optionally receiving, for example by a computer, the cffDNA sequencing reads comprising sequence information from an aneuploid chromosome; (5) dividing the sequence information from the aneuploid chromosome into a plurality of linkage disequilibrium (LD) blocks or genomic windows (GWs); (6) selecting one or more of the sequencing reads corresponding to one or more of the plurality of LD blocks or GWs to produce a set of selected sequencing reads; (8) determining the probability of observing the selected set of sequencing reads under a meiotic-origin model and a under a mitotic-origin model, for example given knowledge of haplotype structure from an external reference panel of phased genome sequences, to produce a set of probability data; and (9) aggregating log-likelihood ratios across the plurality of LD blocks or GWs to produce an aggregated log-likelihood ratio using the set of probability data. In the methods disclosed herein, the meiotic- and the mitotic-origin aneuploidies can be distinguished in that an aggregated log-likelihood ratio significantly greater than zero indicates that the aneuploid chromosome is a meiotic-origin aneuploidy, and an aggregated log-likelihood ratio significantly less than zero indicates that the aneuploid chromosome is a mitotic-origin aneuploidy.

In an initial step of the methods disclosed herein, sequenced fragments that originated from cffDNA are distinguished from those fragments that originated from cfmDNA. This can be achieved analytically by exploiting unique features of the maternal and fetal fragments. By way of example, distinguishing features may include the length of the sequenced fragment, the starting (5′ end) position of the fragment, and/or the ending (3′ end) position of the fragment. These unique features that may distinguish cffDNA from cfmDNA are a consequence of the natural fragmentation of the cell-free DNA as well as its packaging onto nucleosomes relative to cellular DNA. More specifically, in the methods disclosed herein, sequences are identified that originated from cffDNA fragments. In certain embodiments, the at least one distinguishing characteristic that may be used to identify sequencing reads from the cffDNA are selected from aligned coordinates of reads relative to the nearest nucleosome, inferred fragments lengths, methylation patterns, and/or patterns at fragment edges (blunt vs. jagged ends).

In certain embodiments, the cffDNA sequence fragments may be identified using a method called Single reAds Nucleosome-basEd FetAL fraction (SANEFALCON), which was originally introduced to estimate the cffDNA fraction based on genome-wide nucleosome profiles and is disclosed, for example, in Straver, R. et al., Calculating the fetal fraction for noninvasive prenatal testing based on genome-wide nucleosome profiles, Prenatal Diagnosis 2016; 36:614-621. This method is based on the periodic patterns in the depth of coverage (periodicity of 147 bp) that is caused by the nucleosomes. As such, reads where the alignment begins in regions covered by nucleosomes are enriched for cffDNA. As both cffDNA and cfmDNA in maternal plasma is already digested into small fragments by natural processes, no additional shearing may be needed prior to sequencing the fragments, and sequencing reads obtained from both the cffDNA and the cfmDNA fragments may begin at positions where they were cut by natural processes. Based on the understanding that enzymes that cut DNA can easily make cuts in between nucleosomes (e.g., in the areas containing linker DNA), DNA fragments with nucleosomes still attached are largely protected from cutting. Accordingly, it is expected that the distribution in read start positions will change based on the relative amount of large read fragments with respect to the amount of short fragments. The distribution of DNA fragment degradation can then be used to distinguish between the fraction of cffDNA and the fraction of cfmDNA in the maternal sample. Straver reports that cffDNA has start positions that are enriched in nucleosome-covered areas (i.e., any position within the 73 bp upstream or downstream of the mapped nucleosome center), while cfmDNA is enriched in the linker DNA that is outside of the 73 bp upstream or downstream region.

Furthermore, it has also been shown that insert sizes of paired-end reads may also differ between cffDNA and cfmDNA, as disclosed, for example, in Chan, K. A. et al., Size distributions of maternal and fetal DNA in maternal plasma, Clinical Chemistry 2004, 50(1):88-92; Li, Y. et al., Size separation of circulatory DNA in maternal plasma permits ready detection of fetal DNA polymorphisms, Clinical Chemistry 2004, 50(6):1002-1011; and Li, Y. et al., Detection of paternally inherited fetal point mutations for β-thalassemia using size-fractionated cell-free DNA in maternal plasma, JAMA 2005, 293(7):843-849. Thus, in certain embodiments, by combining the nucleosome and insert size signatures, cffDNA fragments of putative fetal origin can be distinguished from cfmDNA fragments of putative maternal origin. Because paired-end reads are widely used for NIPT, for example with software such as the VeriSeq NIPT Solution v.2 from Illumina®, the fragments' length can be deduced, and their position can be intersected with existing maps of nucleosome positioning.

In certain embodiments, after identifying the cffDNA fragments from the cfmDNA fragments, a method for local phasing and imputation of low-coverage sequencing may be applied to the subset of sequence fragments of putative maternal origin. In certain embodiments, the method for local phasing and imputation of low-coverage sequencing may be the GLIMPSE method, as disclosed, for example, in Rubinacci, S. et al., Efficient phasing and imputation of low-coverage sequencing data using large reference panels, Nature Genetics 2021, 53(1):120-126.

In certain embodiments, the local phasing and imputation of cfmDNA may effectively replace the use of a population reference panel with the phased haplotypes of the mother, thereby leveraging the fact that cfmDNA typically constitutes 80-90% of the total cell-free DNA in maternal plasma. Meanwhile, the paternal reference may, in certain embodiments, be based on a population panel, as in the standard implementation of LD-PGTA, or, in certain embodiments, a paternal reference may be based on cell-free DNA obtained from a paternal plasma sample. The LD-PGTA methods can then be applied to the subset of sequences of putative fetal origin to distinguish the meiotic versus mitotic basis of the trisomy. This may be achieved by adapting the statistical models for recent admixture that allow for the use a unique reference panel for each parent, as further detailed, for example, in Ariad, D. et al., Haplotype-aware inference of human chromosome abnormalities, bioRxiv 2021:1-24. Direct knowledge of maternal genotype, may, in certain embodiments, enhance performance of the methods disclosed herein.

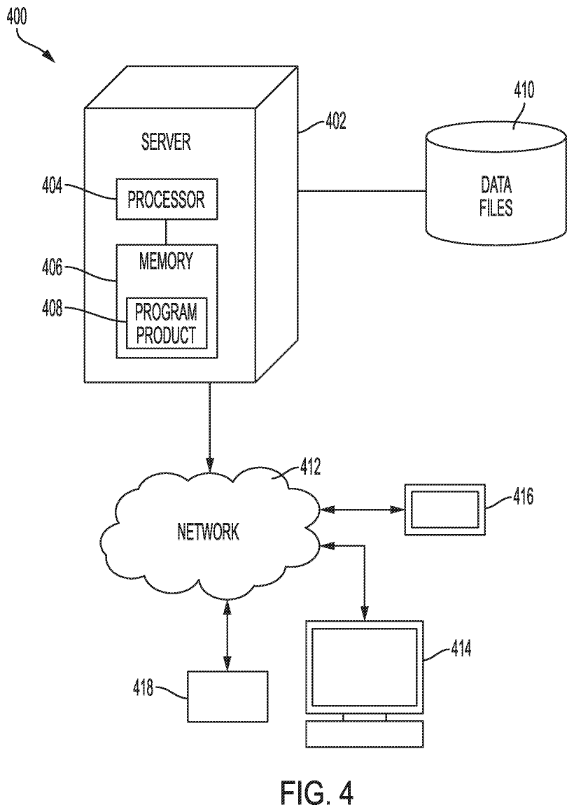

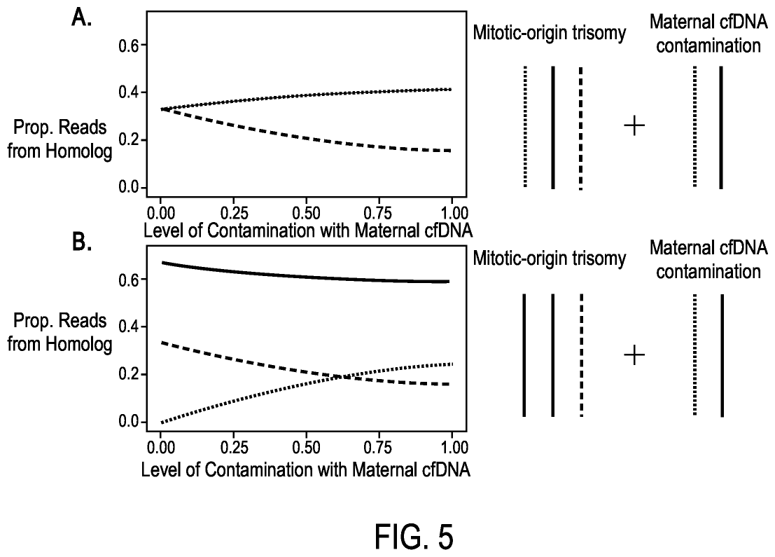

Because the methods disclosed herein (also referred to as “LD-NIPT”) comprise distinguishing cffDNA from cfmDNA, accuracy of the methods will be affected by the accuracy of the distinction. In order to account for a fraction of misclassified reads, the statistical models may in certain embodiments be adjusted by assigning a probability of drawing reads from the maternal homologs, thereby modeling the presence of maternal contamination. In certain embodiments, the rate of maternal contamination can be estimated with existing methods, such as, for example, those disclosed in Straver, R. et al., Calculating the fetal fraction for noninvasive prenatal testing based on genome-wide nucleosome profiles, Prenatal Diagnosis 2016, 36(7):614-621. As shown for example in FIGS. 5A-5B, meiotic- and mitotic-origin aneuploidies may be distinguished based on the probability of reads from each homolog, accounting for a level of contamination with cfmDNA. As shown in FIGS. 5A and 5B, the methods disclosed herein are relatively robust to maternal DNA contamination. FIG. 5 depicts the proportion of simulated reads emanating from each homolog assuming varying rates of cfmDNA contamination under the meiotic trisomy (FIG. 5A) versus mitotic trisomy (FIG. 5B) scenario. Note that even with high rates of cfmDNA contamination, the meiotic and mitotic signatures are distinct, as reflected by the differences between FIG. 5A and FIG. 5B. For simplicity, only mitotic trisomy involving gain of a maternal chromosome is depicted, though the same principles and conclusion apply to mitotic trisomy involving gain of a paternal chromosome.

In certain embodiments disclosed herein, trisomies of meiotic origin produce a unique genetic signature, characterized by the presence of three unique parental haplotypes (two from a single parent) and distinct from the mitotic trisomy signature of only two unique haplotypes chromosome-wide (FIG. 1). Normal gametogenesis produces two genetically distinct copies of each chromosome (i.e., one copy from each parent) that comprise mosaics of two homologs possessed by each parent. Meiotic-origin trisomies may be diagnosed by the presence of one or more tracts with three distinct parental homologs (i.e., transmission of both parental homologs [BPH] from a given parent). In contrast, mitotic-origin trisomies are expected to exhibit only two genetically distinct parental homologs chromosome-wide (i.e., duplication of a single parental homolog [SPH] from a given parent). Triploidy and haploidy will mirror patterns observed for individual meiotic trisomies and monosomies, respectively, but across all 23 chromosome pairs. As shown in FIG. 1, trisomies of meiotic origin may comprise three unique homologs in a centromere-proximal or centromere distal region, while trisomies of mitotic origin may comprise only two unique homologs chromosome-wide. In certain embodiments of the methods disclosed herein, the presence of tracts comprising three unique homologs indicates a meiotic error, which will affect all cells of the fetus and is unambiguously harmful, and in certain embodiments of the methods disclosed herein, the presence of tracts comprising only two unique homologs indicates a mitotic error, which may be compatible with healthy, live birth.

In certain embodiments, the LD-NIPT methods disclosed herein detect cases of haploidy, and in certain embodiments, the LD-NIPT methods disclosed herein detect cases of triploidy.

After probabilistic identification and separation of the cffDNA sequence fragments to identify an aneuploid chromosomes, the present disclosure further provides in certain aspects a statistical method for distinguishing meiotic and mitotic trisomies based on analysis of low-coverage whole-genome sequencing data, as disclosed, for example, in PCT Publication No. WO 2022/098980, entitled Methods and Related Aspects for Analyzing Chromosome Number Status, which is incorporated in its entirety by reference herein.

In some embodiments, the sparse nature of the data may be overcome by leveraging allele frequencies and haplotype patterns (patterns of genetic variation) are correlated in the population and thus observed in a phased population reference panel such as the 1000 Genomes Project. In some embodiments, the sparse nature of the data may be overcome by leveraging allele frequencies and haplotype patterns from the cfmDNA nucleotide sequence fragments obtained from a sample from the mother, such as a blood sample. In certain embodiments, a maternal phased haplotype is used as a reference panel.

Thus, even sparse observations permit probabilistic inferences of genotypes in unobserved parts of the genome. In certain embodiments wherein a population reference panel is used, this may be accomplished through the use of statistical models that describe the probabilities of particular genotype observations, given the patterns of genetic variation observed in a population reference panel (e.g., using publicly available data such as the 1000 Genomes Project or the like). In certain embodiments, a reference panel may be obtained from sequenced nucleotides from the cfmDNA, using a method for local phasing and imputation of low-coverage sequencing, such as GLIMPSE, as disclosed for example in Rubinacci, S. et al., Efficient phasing and imputation of low-coverage sequencing data using large reference panels, Nature Genetics 2021, 53(1):120-126.

In certain embodiments, the methods disclosed herein retain power to distinguish meiotic and mitotic trisomies down to coverage as low as about 0.05× and, in certain embodiments, at higher coverage the methods disclosed herein can also distinguish between meiosis I and meiosis II errors based on centromere-proximal signatures. Together, the methods and related aspects of the present disclosure provide fundamental insight into the mechanistic origins of trisomies during human development, among other applications.

In some exemplary embodiments, meiotic and mitotic trisomies (aneuploidies involving three copies of a chromosome) are discerned based on the unique signatures that they leave within the genotype data. For example, the harmful aneuploidies that arise via meiosis typically generate signatures of three genetically distinct chromosome copies (e.g., both parental homologs (BPH), as denoted for example in FIG. 1). In contrast, the potentially viable mitotic trisomies harbor a signature of only two distinct chromosome copies (wherein two of the three copies are identical (e.g., single parental homologs (SPH), as denoted for example in FIG. 1).

In certain embodiments, the likelihoods of the data under the meiotic trisomy and mitotic trisomy hypothesis may then be contrasted. Typically, the statistical models are generic (e.g., any number of chromosomes involving any combination of identical or distinct homologs can be accommodated), and naturally extend to the related problem of detecting aneuploidies that involve all copies of a chromosome (such as, for example, triploidy or haploidy), which may be missed by previous methods. Optionally, essentially any number of sequencing reads can be accommodated by these methods. The methods disclosed herein also work for subjects of admixed ancestry (e.g., subjects or individuals with recent ancestors from different continental super-populations such as European and East Asian), which accounts for a large proportion of the population. These and other aspects will be apparent upon a complete review of the present disclosure including the accompanying figures.

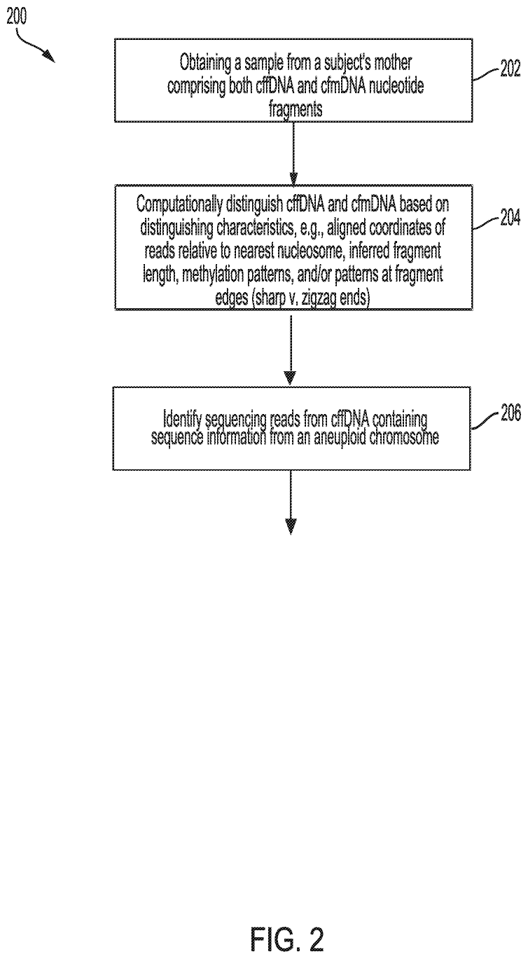

To illustrate certain embodiments of the methods disclosed herein, FIG. 2 is a flow chart that schematically depicts exemplary method steps of identifying by probabilistic separation the cffDNA and cfmDNA, and distinguishing between meiotic- and mitotic-origin aneuploidies in the cffDNA, at least partially using a computer. As shown, method 200 includes obtaining a sample, such as a blood sample, from a subject's mother, wherein the sample comprises both cffDNA and cfmDNA nucleotide fragments (step 202); computationally distinguishing the cffDNA and the cfmDNA based on at least one distinguishing characteristic (e.g., aligned coordinates of reads relative to nucleosome, inferred fragment length, methylation patterns, and/or patterns at fragment edges (blunt v. jagged edges)) (step 204). Based on the separated cffDNA, one may then identify sequence reads from the cffDNA nucleotide fragments that contain sequence information from an aneuploid chromosome (step 206). The methods disclosed herein further comprise receiving, for example by the computer, sequencing reads comprising sequence information from the aneuploid chromosome identified in step 206, and dividing, optionally by the computer, the sequence information from the aneuploid chromosome into a plurality of LD blocks or GWs (step 208). Method 200 also includes selecting, optionally by the computer, one or more of the sequencing reads corresponding to one or more of the plurality of LD blocks or GWs to produce a set of selected sequencing reads (step 210), and determining, optionally by the computer, the probabilities of observing the selected sequencing reads under a meiotic-origin model and under a mitotic-origin model to produce a set of probability data (step 212). In addition, method 200 also includes aggregating, optionally by the computer, log-likelihood ratios across the plurality of LD blocks or GWs to produce aggregated log-likelihood ratio using the set of probability data in which an aggregated log-likelihood ratio significantly greater than zero indicates that the aneuploid chromosome is a meiotic-origin aneuploidy, and wherein an aggregated log-likelihood ratio significantly less than zero indicates that the aneuploid chromosome is a mitotic-origin aneuploidy (step 214). In these contexts, significance may be defined, for example, by a 95% confidence interval that does not overlap zero. In some embodiments, this corresponding 95% confidence interval is constructed by taking the 2.5% and 97.5% quantiles of the bootstrap distribution. Any confidence level, however, can optionally be chosen depending on desired sensitivity vs. specificity.

In some embodiments, a probability that two sequencing reads are obtained from an identical haplotype under the meiotic-origin model is about ⅓. In some embodiments, a probability that two sequencing reads are obtained from an identical haplotype under the mitotic-origin model is about 5/9. In some embodiments, the sequence information comprises a coverage of between about 0.05× and about 0.5×.

In some embodiments, the aneuploid chromosome comprises a trisomy. In some embodiments, when the aneuploid chromosome is a meiotic-origin aneuploidy, the method further comprises determining whether an aneuploidy is due to a meiosis I error or a meiosis II error. In some embodiments, the method comprises determining a significance of the aggregated log-likelihood ratio using at least one statistical procedure (e.g., a bootstrap or weighted jackknife procedure or the like).

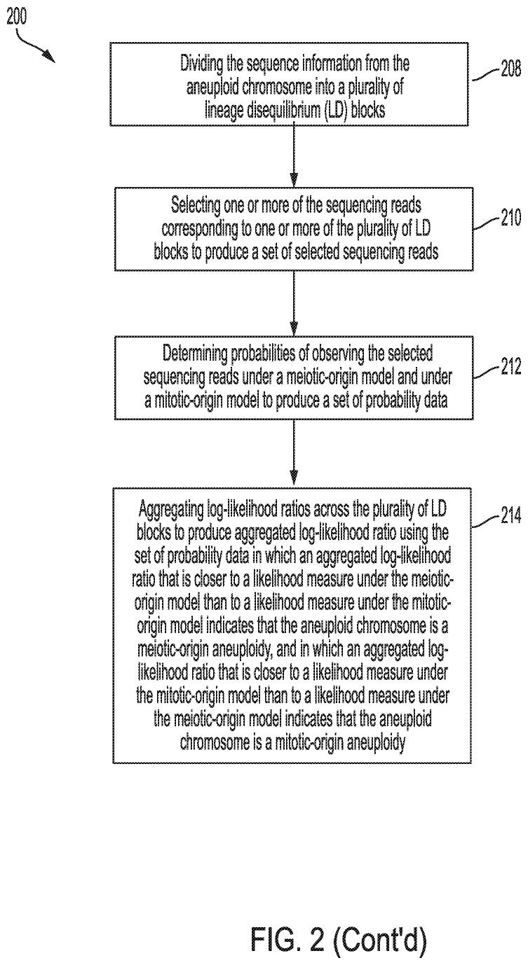

To further illustrate, FIGS. 3A-3D schematically depicts exemplary method steps of distinguishing between meiotic- and mitotic-origin aneuploidies. As shown, one exemplary method includes, within defined LD blocks or GWs, select reads overlapping informative single nucleotide polymorphs (SNPs) that tag common haplotype variation (FIG. 3A). The method may also include obtaining joint frequencies of corresponding SNPs from a phased panel of reference haplotypes, such as ancestry-matched reference haplotypes or from a reference maternal haplotype (FIG. 3B). The method may also include randomly selecting multiple reads, such as 2-16 reads, and computing probabilities of observed alleles under competing trisomy hypotheses (FIG. 3C). In addition, the method may also include comparing the hypotheses by computing a likelihood ratio and estimating the mean and variance by sub-sampling random sets of reads using a bootstrapping approach (FIG. 3D).

Further disclosed herein are methods of classifying a chromosome number status of a test sample, e.g., the sample of cffDNA, typically at least partially using a computer. In certain embodiments, the methods disclosed herein comprise selecting sequencing reads (e.g., cffDNA sequence fragments) that comprise nucleic acid variants within defined genomic windows to produce sets of observed cffDNA nucleic acid variants. The methods disclosed herein may further comprise obtaining joint allele frequencies and/or linkage disequilibrium patterns of corresponding nucleic acid variants observed in a reference subject population or from the cfmDNA nucleotide fragments to produce sets of reference subject joint allele frequency and/or linkage disequilibrium pattern data. In addition, the methods may further comprise classifying the chromosome number status of the cffDNA using the sets of observed cffDNA nucleic acid variants and the sets of reference subject (e.g., maternal) joint allele frequency and/or linkage disequilibrium pattern data.

In some embodiments, the nucleic acid variants comprise single nucleotide variants (SNVs), insertions or deletions (indels), gene fusions, copy number variants (CNVs), transversions, translocations, frame shifts, duplications, epigenetic variants, and repeat expansions.

In some embodiments, the methods disclosed herein further include performing whole genome sequencing of nucleic acids obtained from the cffDNA nucleotide fragments to produce the sequencing reads. In some embodiments, the sequencing reads comprise a coverage of less than about 2×, less than about 1×, less than about 0.50×, less than about 0.25×, less than about 0.15×, less than about 0.10×, or less than about 0.05× of a genome of the fetus from which the cffDNA originated. In some embodiments, the chromosome number status comprises a state selected from the group consisting of: a monosomy, a monoploidy, a haploidy, a disomy, a diploidy, a trisomy, a triploidy, a tetrasomy, a tetraploidy, a pentasomy, a pentaploidy, and a mosaicisim. In some embodiments, the chromosome number status comprises a meiotic-origin aneuploidy, whereas in other embodiments, the chromosome number status comprises a mitotic-origin aneuploidy. In some embodiments, the methods include determining one or more both parental homologs (BPH) and/or one or more single parental homolog (SPH) signatures for the subject.

In some embodiments, the methods include randomly resampling two or more of the sequencing reads in the set of observed cffDNA nucleic acid variants to produce one or more resampled alleles for each of the defined genomic windows. In some embodiments, the methods include randomly resampling between about 2 and about 1000 of the sequencing reads, between about 3 and about 100 of the sequencing reads, between about 4 and about 50 of the sequencing reads, between about 5 and about 30 of the sequencing reads, or between about 6 and about 20 of the sequencing reads. In some embodiments, the methods include computing likelihood distributions of the resampled alleles under at least two competing chromosome number status hypotheses for each of the defined genomic windows. In some embodiments, the methods include computing the likelihood distributions of the resampled alleles using one or more statistical models. In some embodiments, the methods include comparing the competing chromosome number status hypotheses by computing a log likelihood ratio for each of the defined genomic windows. In some embodiments, the methods include estimating a mean value and a variance value by resampling random sets of sequencing reads using at least one bootstrapping approach for each of the defined genomic windows to produce a set of bootstrap distributions. In some embodiments, the methods include combining the log likelihood ratios from multiple defined genomic windows to produce a combined log likelihood ratio. In some embodiments, the methods include estimating a confidence interval using a mean value and a variance value for the combined log likelihood ratio.

In some embodiments, the defined genomic windows are non-overlapping. In some embodiments, the defined genomic windows comprise between about 2 and about 100,000 defined genomic windows, between about 3 and about 10,000 defined genomic windows, between about 4 and about 1,000 defined genomic windows, between about 5 and about 100 defined genomic windows, between about 10 and about 75 defined genomic windows, between about 20 and about 50 defined genomic windows, or between about 30 and about 40 defined genomic windows. In some embodiments, a given defined genomic window comprises a length of between about 5 bases and about 1,000,000 bases, between about 10 bases and about 100,000 bases, between about 100 bases and about 10,000 bases, or between about 500 bases and about 1,000 bases.

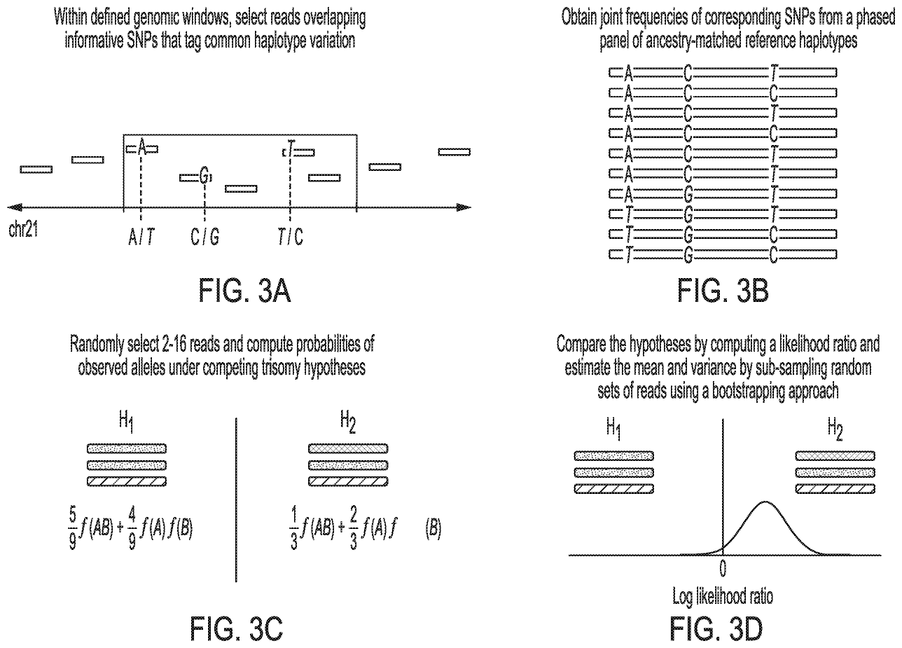

The present disclosure also provides various systems and computer program products or machine readable media for use with the methods disclosed herein. In some aspects, for example, the methods described herein are optionally performed or facilitated at least in part using systems, distributed computing hardware and applications (e.g., cloud computing services), electronic communication networks, communication interfaces, computer program products, machine readable media, electronic storage media, software (e.g., machine-executable code or logic instructions) and/or the like. To illustrate, FIG. 4 provides a schematic diagram of an exemplary system suitable for use with implementing at least some aspects of the methods disclosed in this application. As shown, system 400 includes at least one controller or computer, e.g., server 402 (e.g., a search engine server), which includes processor 404 and memory, storage device, or memory component 406, and one or more other communication devices 414, 416, (e.g., client-side computer terminals, telephones, tablets, laptops, other mobile devices, etc. (e.g., for receiving captured images and/or videos for further analysis, etc.)) positioned remote from camera device 418, and in communication with the remote server 402, through electronic communication network 412, such as the Internet or other internetwork. Communication devices 414, 416 typically include an electronic display (e.g., an internet enabled computer or the like) in communication with, e.g., server 402 computer over network 412 in which the electronic display comprises a user interface (e.g., a graphical user interface (GUI), a web-based user interface, and/or the like) for displaying results upon implementing the methods described herein. In certain aspects, communication networks also encompass the physical transfer of data from one location to another, for example, using a hard drive, thumb drive, or other data storage mechanism. System 400 also includes program product 408 (e.g., related to implementing a method of distinguishing between meiotic- and mitotic-origin aneuploidies as described herein) stored on a computer or machine readable medium, such as, for example, one or more of various types of memory, such as memory 406 of server 402, that is readable by the server 402, to facilitate, for example, a guided search application or other executable by one or more other communication devices, such as 414 (schematically shown as a desktop or personal computer). In some aspects, the system 400 optionally also includes at least one database server, such as, for example, server 410 associated with an online website having data stored thereon (e.g., entries corresponding to more reference panels, etc.) searchable either directly or through search engine server 402. System 400 optionally also includes one or more other servers positioned remotely from server 402, each of which are optionally associated with one or more database servers 410 located remotely or located local to each of the other servers. The other servers can beneficially provide service to geographically remote users and enhance geographically distributed operations.

As understood by those of ordinary skill in the art, memory 406 of the server 402 optionally includes volatile and/or nonvolatile memory including, for example, RAM, ROM, and magnetic or optical disks, among others. It is also understood by those of ordinary skill in the art that although illustrated as a single server, the illustrated configuration of server 402 is given only by way of example and that other types of servers or computers configured according to various other methodologies or architectures can also be used. Server 402 shown schematically in FIG. 4, represents a server or server cluster or server farm and is not limited to any individual physical server. The server site may be deployed as a server farm or server cluster managed by a server hosting provider. The number of servers and their architecture and configuration may be increased based on usage, demand and capacity requirements for the system 400. As also understood by those of ordinary skill in the art, other user communication devices 414, 416 in these aspects, for example, can be a laptop, desktop, tablet, personal digital assistant (PDA), cell phone, server, or other types of computers. As known and understood by those of ordinary skill in the art, network 412 can include an internet, intranet, a telecommunication network, an extranet, or world wide web of a plurality of computers/servers in communication with one or more other computers through a communication network, and/or portions of a local or other area network.

As further understood by those of ordinary skill in the art, exemplary program product or machine readable medium 408 is optionally in the form of microcode, programs, cloud computing format, routines, and/or symbolic languages that provide one or more sets of ordered operations that control the functioning of the hardware and direct its operation. Program product 408, according to an exemplary aspect, also need not reside in its entirety in volatile memory, but can be selectively loaded, as necessary, according to various methodologies as known and understood by those of ordinary skill in the art.

As further understood by those of ordinary skill in the art, the term “computer-readable medium” or “machine-readable medium” refers to any medium that participates in providing instructions to a processor for execution. To illustrate, the term “computer-readable medium” or “machine-readable medium” encompasses distribution media, cloud computing formats, intermediate storage media, execution memory of a computer, and any other medium or device capable of storing program product 408 implementing the functionality or processes of various aspects of the present disclosure, for example, for reading by a computer. A “computer-readable medium” or “machine-readable medium” may take many forms, including but not limited to, non-volatile media, volatile media, and transmission media. Non-volatile media includes, for example, optical or magnetic disks. Volatile media includes dynamic memory, such as the main memory of a given system. Transmission media includes coaxial cables, copper wire and fiber optics, including the wires that comprise a bus. Transmission media can also take the form of acoustic or light waves, such as those generated during radio wave and infrared data communications, among others. Exemplary forms of computer-readable media include a floppy disk, a flexible disk, hard disk, magnetic tape, a flash drive, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave, or any other medium from which a computer can read.

Program product 408 is optionally copied from the computer-readable medium to a hard disk or a similar intermediate storage medium. When program product 408, or portions thereof, are to be run, it is optionally loaded from their distribution medium, their intermediate storage medium, or the like into the execution memory of one or more computers, configuring the computer(s) to act in accordance with the functionality or method of various aspects. All such operations are well known to those of ordinary skill in the art of, for example, computer systems.

To further illustrate, in certain aspects, this application provides systems that include one or more processors, and one or more memory components in communication with the processor. The memory component typically includes one or more instructions that, when executed, cause the processor to provide information that causes sequence information, related data, and/or the like to be displayed (e.g., upon being received from nucleic acid sequencing device 418 and/or via communication devices 414, 416 or the like) and/or receive information from other system components and/or from a system user (e.g., via nucleic acid sequencing device 418 and/or via communication devices 414, 416, or the like).

In some aspects, program product 408 includes non-transitory computer-executable instructions which, when executed by electronic processor 404 perform at least: selecting within defined genomic windows, reads overlapping informative SNPs that tag common haplotype variation; obtaining joint frequencies of corresponding SNPs from a phased panel of ancestry-matched reference haplotypes; randomly selecting multiple (e.g., 2-16) sequencing reads; computing probabilities of observed alleles under competing trisomy hypotheses; and comparing the hypotheses by computing a likelihood ratio and estimating a mean and variance by sub-sampling random sets of reads using a bootstrapping approach. Other exemplary executable instructions that are optionally performed are described further herein.

Additional details relating to computer systems and networks, databases, and computer program products are also provided in, for example, Peterson, Computer Networks: A Systems Approach, Morgan Kaufmann, 5th Ed. (2011), Kurose, Computer Networking: A Top-Down Approach, Pearson, 7^{th} Ed. (2016), Elmasri, Fundamentals of Database Systems, Addison Wesley, 6th Ed. (2010), Coronel, Database Systems: Design, Implementation, & Management, Cengage Learning, 11^{th} Ed. (2014), Tucker, Programming Languages, McGraw-Hill Science/Engineering/Math, 2nd Ed. (2006), and Rhoton, Cloud Computing Architected: Solution Design Handbook, Recursive Press (2011), which are each incorporated by reference in their entirety.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, devices, systems, computer readable media, and/or component parts or other aspects thereof can be used in various combinations. All patents, patent applications, websites, other publications or documents, and the like cited herein are incorporated by reference in their entirety for all purposes to the same extent as if each individual item were specifically and individually indicated to be so incorporated by reference.

1. A method of distinguishing between meiotic- and mitotic-origin aneuploidies of a subject at least partially using a computer, the method comprising:
obtaining non-invasive prenatal testing (NIPT) sequencing data from a sample from the subject's mother, the sample comprising cell-free fetal DNA (cffDNA) nucleotide fragments originating from the subject and cell-free maternal DNA (cfmDNA) nucleotide fragments originating from the mother;
identifying, via probabilistic separation using the computer, the cffDNA nucleotide fragments in the sample from the cfmDNA nucleotide fragments based on at least one distinguishing characteristic;
identifying sequencing reads from the cffDNA nucleotide fragments that comprise sequence information from an aneuploid chromosome;
dividing the sequencing reads from the aneuploid chromosome into a plurality of linkage disequilibrium (LD) blocks or genomic windows (GWs);
selecting one or more of the sequencing reads corresponding to one or more of the plurality of LD blocks or GWs to produce a set of selected sequencing reads;
determining probabilities of observing the selected set of sequencing reads under a meiotic-origin model and under a mitotic-origin model to produce a set of probability data; and,
aggregating log-likelihood ratios across the plurality of LD blocks or GWs to produce an aggregated log-likelihood ratio using the set of probability data, wherein an aggregated log-likelihood ratio significantly greater than zero indicates that the aneuploid chromosome is a meiotic-origin aneuploidy, and wherein an aggregated log-likelihood ratio significantly less than zero indicates that the aneuploid chromosome is a mitotic-origin aneuploidy, thereby distinguishing between the meiotic- and the mitotic-origin aneuploidies.

2. The method according to claim 1, wherein the at least one distinguishing characteristic is chosen from aligned coordinates of reads relative to the nearest nucleosome, inferred fragments lengths, methylation patterns, or patterns at fragment edges.

3. The method according to claim 1, wherein the set of selected sequencing reads comprise overlapping informative single nucleotide polymorphs (SNPs) that tag common haplotype variation.

4. The method according to claim 1, further comprising obtaining joint frequencies of corresponding SNPs from a phased reference haplotype panel.

5. The method according to claim 4, wherein the phased reference haplotype panel is obtained from the cfmDNA nucleotide fragments or from a population reference panel.

6. (canceled)

7. The method according to claim 1, wherein the probabilities of observing the selected set of sequencing reads under a meiotic-origin model and under a mitotic-origin model is determined through the use of haplotype structure from an external reference panel of phased genome sequences.

8. The method according to claim 1, wherein the subject is an in utero fetus.

9. The method according to claim 1, wherein the sequencing reads from the cffDNA nucleotide fragments that comprise sequence information from an aneuploid chromosome are received by a computer, and wherein the sequencing reads from the aneuploid chromosome are divided by the computer into the plurality of LD blocks or GWs.

10. The method according to claim 1, wherein the sample from the subject's mother is a blood serum sample.

11. The method according to claim 1, wherein the aneuploid chromosome comprises a trisomy.

12. The method according to claim 1, further comprising determining whether an aneuploidy is due to a meiosis I error or a meiosis II error when the aneuploid chromosome is a meiotic-origin aneuploidy.

13. The method according to claim 1, further comprising determining a significance of the aggregated log-likelihood ratio using at least one statistical procedure.

14. The method of claim 13, wherein the statistical procedure comprises a bootstrap or weighted jackknife procedure.

15. The method according to claim 1, wherein the sequencing reads from the cffDNA nucleotide fragments comprise a coverage of less than about 2×, less than about 1×, less than about 0.50×, less than about 0.25×, less than about 0.15×, less than about 0.10×, or less than about 0.05× of a genome of a fetus.

16. The method according to claim 1, wherein a probability that two sequencing reads are obtained from an identical haplotype under the meiotic-origin model is about ⅓ or wherein a probability that two sequencing reads are obtained from an identical haplotype under the mitotic-origin model is about 5/9.

17. (canceled)

18. A system, comprising at least one controller that comprises, or is capable of accessing, computer readable media comprising non-transitory computer executable instructions which, when executed by at least one electronic processor, perform at least:
receiving NIPT sequencing data from a sample from the subject's mother, the sample comprising cell-free fetal DNA (cffDNA) nucleotide fragments originating from the subject and cell-free maternal DNA (cfmDNA) nucleotide fragments originating from the mother;
identifying, via probabilistic separation, cffDNA nucleotide fragments in the sample from the cfmDNA nucleotide fragments based on at least one distinguishing characteristic;
identifying sequencing reads obtained from cffDNA nucleotide fragments comprising sequence information from an aneuploid chromosome;
dividing the sequence information from the aneuploid chromosome into a plurality of linkage disequilibrium (LD) blocks or genomic windows (GWs);
selecting one or more of the sequencing reads corresponding to one or more of the plurality of LD blocks or GWs to produce a set of selected sequencing reads;
determining probabilities of observing the selected set of sequencing reads under a meiotic-origin model and under a mitotic-origin model to produce a set of probability data; and,
aggregating log-likelihood ratios across the plurality of LD blocks or GWs to produce aggregated log-likelihood ratio using the set of probability data, wherein an aggregated log-likelihood ratio significantly greater than zero indicates that the aneuploid chromosome is a meiotic-origin aneuploidy, and wherein an aggregated log-likelihood ratio significantly less than zero indicates that the aneuploid chromosome is a mitotic-origin aneuploidy.

19. A computer readable media comprising non-transitory computer executable instructions which, when executed by at least electronic processor, perform at least:
receiving NIPT sequencing data from a sample from the subject's mother, the sample comprising cell-free fetal DNA (cffDNA) nucleotide fragments originating from the subject and cell-free maternal DNA (cfmDNA) nucleotide fragments originating from the mother;
identifying, via probabilistic separation, cell-free fetal DNA (cffDNA) nucleotide fragments in the sample from the cfmDNA nucleotide fragments based on at least one distinguishing characteristic;
identifying sequencing reads obtained from cffDNA nucleotide fragments comprising sequence information from an aneuploid chromosome;
dividing the sequence information from the aneuploid chromosome into a plurality of linkage disequilibrium (LD) blocks or genomic windows (GWs);
selecting one or more of the sequencing reads corresponding to one or more of the plurality of LD blocks or GWs to produce a set of selected sequencing reads;
determining probabilities of observing the selected set of sequencing reads under a meiotic-origin model and under a mitotic-origin model to produce a set of probability data; and,
aggregating log-likelihood ratios across the plurality of LD blocks or GWs to produce aggregated log-likelihood ratio using the set of probability data, wherein an aggregated log-likelihood ratio significantly greater than zero indicates that the aneuploid chromosome is a meiotic-origin aneuploidy, and wherein an aggregated log-likelihood ratio significantly less than zero indicates that the aneuploid chromosome is a mitotic-origin aneuploidy.

20. The system of claim 18, wherein the sequencing reads from the cffDNA nucleotide fragments comprise a coverage of less than about 2×, less than about 1×, less than about 0.50×, less than about 0.25×, less than about 0.15×, less than about 0.10×, or less than about 0.05× of a genome of a fetus.

21. The system of claim 18, wherein the instructions further perform obtaining joint frequencies of corresponding SNPs from a phased reference haplotype panel.

22. The system of claim 21, wherein the phased reference haplotype panel is obtained from cfmDNA, wherein the cfmDNA originated from a sample comprising both the cffDNA and cfmDNA.
